(19) United States
(12) Patent Application Publication
(10) Pub. No.: US 2009/0220612 A1
(43) Pub. Date: Sep. 3, 2009

(54) HOLLOW-FIBRE-BASED BIOCOMPATIBLE DRUG DELIVERY DEVICE WITH ONE OR MORE LAYERS

(76) Inventor: Semali Priyanthi Perera, Bath (GB)

Correspondence Address: BUCHANAN, INGERSOLL & ROONEY PC, POST OFFICE BOX 1404, ALEXANDRIA, VA, 22313-1404, US

(21) Appl. No.: 12/092,537

(22) PCT Filed: Nov. 3, 2006
(86) PCT No.: PCT/GB2006/004110

§ 371 Date: Aug. 8, 2008

(30) Foreign Application Priority Data
Nov. 4, 2005 (GB) 0522569.3

(51) Int. Cl. A61K 9/50

(52) U.S. Cl. 424/497

(57) ABSTRACT

A biocompatible drug delivery device in which the mean pore size in one or more layers is less than 100 μm. The device may be a hollow fibre or a membrane comprising a number of hollow fibres or a microsphere. The invention also extends to a method for preparing porous hollow fibres or microspheres, to the apparatus for preparing said fibres and to the use of the fibres as drug delivery devices.

The present invention is directed towards the preparation of biocompatible drug delivery devices. Specifically it is directed towards the production of hollow fibres, in particular fibres of nanoporosity without additional coating and a generic synthesis route for the production of a range of hollow fibres with specific properties directed towards the delivery of drugs. Further, the invention is directed towards the use of such fibres to release compositions in a controlled fashion as the fibres degrade. The invention is also directed towards the preparation of microbeads as drug delivery devices and in particular to a synthesis route for the production of microbeads with specific properties.

Although chemotherapy can be given orally, most drugs used to treat cancer are given intravenously. The chemotherapy is either injected directly into a vein or through a thin tube called a venous catheter, a tube temporarily put into a large vein of the heart to make injections easier. However, there are many problems associated with this. Firstly, because therapeutics are commonly delivered intravenously, a relatively high dose needs to be administered. Consequently it can cause clotting and so adjustments of dose may be needed to prevent thrombosis. Secondly, many people also experience substantial side effects from the treatment because the drugs used affect normal cells as well as cancer cells. Such side effects include mucositis, immunosuppression, nausea/vomiting and hair loss. Thirdly, several courses of chemotherapy may be required to identify an active drug. There is therefore an enormous medical need for a means to enable sustained local delivery of chemotherapeutics to reduce side effects and improve quality of life

It is therefore an object of the present invention to prepare biocompatible drug delivery devices (which may be hollow fibres or microbeads) which can carry drugs for controlled release. The concept is based on the use of biodegradable, implantable devices which are capable of releasing one or a number of drugs over a period of time as the devices (which act as capsules) degrade. This device can either be connected to an inlet port on the surface of the body, thus allowing flushing of the devices and investigation into appropriate treatments/doses, or can be used as a stand alone “stent” implanted within a patient. The device also can be injected into a cavity of the patient as a microemulsion.

As well as treatment of cancers, this device could revolutionise the treatment of a number of diseases/disorders which typically require long-term attention. It is a further object of the present invention to enable the provision of effective, convenient, low cost treatment of chronic diseases. It will benefit patients by giving them peace of mind that the treatment is targeted at the problem area, will reduce side effects, and will be more effective and rapid than other treatments. Hospitals will benefit by the reduction in total cost of treatment and drain on resources.

According to a first aspect of the present invention, there is provided a biocompatible drug delivery device in which the mean pore size in one or more layers is less than 100 μm and in which a drug is carried. Such devices can be produced economically and reliably i.e. without defects, and are useful to a range of situations where devices with a small pore size, range of wall thickness and a controlled degradation rate are required to prevent dose dumping.

The device may be a hollow fibre of one or more layers. The mean pore size may be controlled to be in the claimed range in the outer wall and this may optionally be less than 50 μm, 10 μm, 1 μm, 100 nm or even less than 10 nm. The porosity may be even throughout the cross section of the wall of the fibre or may vary across the fibre. In some cases there will be different porosities across the cross section of the fibre. In particular, there may be a higher porosity towards the centre of the fibre, and a lower porosity in the outer layers to provide the strength of fibre aligned with the drug delivery properties desired.

According to a further aspect of the present invention, the device may be a microsphere. The porosity may vary across the radius of the sphere or may be substantially even. There may be a higher porosity towards the centre of the sphere and a lower porosity towards the outer surface. This variable porosity and the thickness of the respective layers play a significant role in the speed of release of the drug from the device.

The fibre or microsphere may be biodegradable such that it will decompose substantially completely over a period of 30 days, 60 days, 90 days, 120 days or anything from 1-24 months. The fibre or sphere may consist of two or more layers, in which different drug compositions may be carried within each layer or within the lumen of the fibre or macrovoids of the sphere, such that the medication applied varies over time as the fibre or sphere decomposes. The different compositions may be different chemical formulations or may alternatively be different concentrations of the same drug.

The fibre or microsphere may be an organic or polymeric fibre or sphere comprising a polymer, an additive and one or more drug compositions. The fibre may include additional drugs in different layers or contained within the lumen (hollow) or macrovoids to be released in a controlled fashion over a period of time. Details of preferred drug compositions and concentrations may be found in the following examples and in the claims.

An advantage to the production of double or triple layer hollow fibres (in addition to an increased mechanical strength which may be beneficial in avoiding breaking or dose dumping the drug delivery device when implanting it in to the patient) is that the fibres are largely defect free. With two or three layers of the same polymeric composition forming the scaffold for the drugs, any defects in one layer are extremely unlikely to be mirrored by a similar defect in the next layer. The net effect is that there are no pinholes which pass through the fibre and it can therefore be used as a scaffold for a drug delivery device without the risk of dose dumping through a defect.

Further, as discussed above, it is possible to have different compositions or different concentrations in the two or more layers as well as a drug contained within the lumen. It is therefore possible to produce a fibre where each layer is tailored towards a particular property or treatment which may vary over time.

A further aspect of the present invention is the high surface to volume ratio of the biocompatible porous hollow fibre or microsphere which may be greater than 1,000 m²/m³ for each delivery device. The area to volume ratio may be in the range 1,000-30,000 m²/m³, preferably 1,000-6,000 m²/m³, and most preferably 4,000-6,000 m²/m³.

The hollow fibres may be arranged in an appropriate configuration for the specific medical application such that they have the desired drug release properties. In particular, the fibres may be arranged such that when they degrade and start to release the drug or drugs, these are targeted at the intended area (e.g. tumour or damaged tissue). In a first arrangement, one or more, optionally 5, 10, 20 or more fibres are bundled together. These may be held tightly in a cylindrical configuration or constrained to another shape which maximises the effectiveness of the delivery device for the release of drugs. The fibres may be held together by a casing which may or may not be biodegradable.

As discussed above, the hollow fibres may contain the same or different drugs in the same or different concentrations within the layer or layers of the fibre and in the hollow or lumen of each fibre. Further, the drugs in some or all of the layers and lumen may be microbead encased to enable a double time-release mechanism to be created. Such microbeads may have an average dimension in the range of 10-50 μm, and are preferably of a uniform diameter. For example, in one embodiment at least 90% of the microbeads have a diameter of n±1 μm where n=10-50 μm. These microbeads in the hollow fibre may be beads produced by known techniques, for example by emulsion techniques, or may be beads or microspheres produced according to the present invention.

Each hollow fibre may, in addition, have an extra outer layer provided to avoid premature drug release. The individual fibres may also be selected to allow the configuration to act as a stand-alone implant or stent. The fibres are sealed at each end and the drugs are released by a combination of diffusion of the drug through the walls of the fibre and degradation of the wall. In stents, some layers of the fibre are developed to include a stronger polymer (for example, polylactide (PLA)) to maintain the strength, integrity and flexibility of the coil. This may have application in the treatments of colon or oesophagus type cancers.

Alternatively, one or both ends of each fibre may be connected to the surface of the body of the patient to allow the fibres to be flushed and refilled with drugs. This connection may be direct, or indirect via an outlet port, lumen or hickman line. The device may be flushed with any suitable material, for example saline or heparin, and the replacement drugs may be the same or different to those previously present. This may have particular application when trying to establish a treatment regime in the early stages of chemotherapy, and different drugs or concentrations of drugs need to be tried to determine what is most effective.

The fibres may be woven together in a patch-like configuration which may then be placed or attached to the site of interest. The patch may be attached, for example, with surgical staples. The patch may be configured to be in an appropriate shape for the site of interest, for example, a ring or sphere to encase part or all of the site of interest. Alternatively, the fibres may be prepared such that they are helical and can be used as a stent within narrow orifices such as the throat (oesophagus), arteries, colon, bowel, ovaries etc. The helical arrangement allows the orifice to be kept open while allowing the drug to be delivered directly to the site of interest.

The biocompatible porous hollow fibre or microsphere may include a high percentage of drugs to be delivered. According to one embodiment, there is at least 5% by weight of drugs in the polymer solution used to prepare the delivery device, although values of up to 80% or higher may also be present. The polymer solution used to prepare the delivery device may therefore have 5-80% by weight of drugs, or 10-75%, 20-70%, 30-65%, 40-80% or 50-55% as appropriate for the situation. This will depend on the nature of the drugs being released and the desired speed of delivery.

The level of drug entrapped within a device (hollow fibre or microsphere) or loaded in the drug delivery device will depend on the encapsulation efficiency which may be from 10-80%, typically 10-40% or 15-30%. This may produce fibres or microspheres having a drug loading of 1-50% by weight or 1-45%, 2-25%, 3-20% or 4-15% by weight.

The outer diameter of the biocompatible fibres produced can be 10-200 μm depending on the diameter of the spinneret and the number of layers used. Therefore, lightweight and compact drug delivery devices can be made using a single hollow fibre or a cluster of narrower fibres as appropriate. The hollow fibres are nanoporous or microporous and can be tailored to exhibit significant drug pharmacokinetics, bending strength (flexibility) and liquid bursting pressure (1-7 bar). The properties of the fibre can be tailored to individual situations.

As indicated above, the microspheres according to the present invention may have a diameter of 10-70 μm, preferably 20-40 μm or 25-35 μm or 35-50 μm or 50-70 μm depending on the particular circumstances. The microspheres of the present invention are generally produced to be of a uniform size and a very narrow size distribution.

According to another aspect of the present invention, there is provided a method for preparing biocompatible porous hollow fibres, in which a spinning dope is prepared in a viscous liquid or gel form, filtered using a mesh, the dope is degassed in a piston delivery vessel attached to a spinneret, the vessel is pressurised using an inert gas using jets, the dope is extruded through the spinneret to form a fibre precursor, the precursor is thoroughly washed to remove any residual solvent and dried. All the equipment is sterilised before use, preferably by steam sterilisation.

The hollow fibres formed by the method of the present invention are the result of the controlled solidification process. First a spinning mixture or dope is prepared from a polymer, a solvent, optionally a binder/additive and any drug composition(s) to be included within the layers of fibre. Subsequently, the produced mixture is extruded through a spinneret into a bath of non-solvent. This non-solvent, selected from a number of internal coagulants including distilled water, is also introduced through the bore of the spinneret. Exchange of solvent and non-solvent leads to thermodynamic instability of the spinning mixture and induces liquid-liquid demixing. Further exchange leads to solidification of the polymer-rich phase. The precursor is washed and dried to remove any residual solvent. Subsequently, a further drug composition may be added to the lumen (hollow) of the produced fibre through a syringe pump.

The produced compact fibres show very good quality and may have different porosities across the cross section of the fibre with a preferred total porosity in the range 30-55%, in particular 35-45%. Average pore size and effective surface porosity of the hollow fibres can be determined by the Poiseuille flow method. In one embodiment, the fibres produced have a pore size in the range 10 m≦pore diameter≦0.1 mm. In another embodiment, the fibres produced have a pore size in the range 100 nm<pore diameter<1 μm. In a still further embodiment, the fibres produced have a pore size in the range pore diameter≦1 nm.

According to another aspect of the present invention, there is provided a method for preparing biocompatible microspheres in which: a polymer is dissolved in a suitable solvent; a drug is dissolved in or dispersed in an organic solvent containing the polymer; the mixture is fed to a high pressure airbrush or spray device with a small nozzle; and the mixture is sprayed under water and microspheres are formed by solvent de-mixing. Similarly, the mixture may be fed to a ceramic hollow fibre bundle with 2-20 nm pores to create micro droplets under water. The feed pressures could be from 2-10 bar.

According to a further aspect of the present invention, there is provided a method for preparing biocompatible microspheres in which: a polymer is dissolved in a suitable solvent; a drug is dissolved in or dispersed in an organic solvent containing the polymer; the mixture is fed to a high pressure airbrush with a small nozzle; and the mixture is sprayed into an antistatic chamber with water saturated air and the solvent is extracted from the produced droplets to form microspheres.

In each case, the microspheres are removed and dried to remove substantially all of the solvent such that the microsphere may contain 0-2% by weight of solvents.

The size and density of the microspheres is controlled and affected by the method used, the spraying pressure and the air gap (gap where beads are exposed to air before polymer solidification or phase inversion) or pore size of the fibres used. In each case, the spheres produced have macrovoids to accommodate more drugs to be delivered.

Although the hollow fibres or microspheres do not have to be biodegradable to operate as drug delivery devices, to be most effective they would degrade in the body over time in order to enable more practical control of the dose of drugs over time (and to prevent any implant from causing inflammation or requiring further surgery for removal).

The fibres or microspheres may be made primarily from any biodegradable material, i.e., where over time the material will decompose mainly to either CO₂ or water or harmless acid. For example: Polyglycolide (PGA) where the degradation product is glycolic acid (a natural metabolite which may be eliminated from the body through the Krebs cycle) or Poly(L-lactic) acid (PLA) where the degradation product is lactic acid (which is eventually converted to carbon dioxide and water by the Krebs cycle and released through respiration).

Examples of suitable materials for the production of the biocompatible fibres or microspheres include: Poly(ethylene terephathalate) (which resists fungal and enzymatic degradation) with the addition of 20-25% PLA (which introduces the biodegradation properties); PLA; PGA; a copolymer of PLA and PGA to form PLGA (poly lactic co glycolic acid); Lactide-glycolide copolymers (PLG); Poly-ε-caprolactone (PCL); Lactide-caprolactone copolymers; and Cellulose-based polymers.

Further biocompatible materials that may be suitable include: Acrylate polymers and copolymers: (for example methyl methacrylate, methacrylic acid; hydroxyalkyl acrylates and methacrylates; methylene glycol dimethacrylate; acrylamide, bisacrylamide); Ethylene glycol polymers and copolymers; Oxyethylene and oxypropylene polymers; Poly(vinyl alcohol) and Polyvinylacetate; Polyvinylpyrrolidone and polyvinylpyridine.

Other possible materials include Biodegradable polyphosphazenes, Pseudo-poly(amino acids), polyethylene glycol containing poly-carbonates, phosphorous containing biodegradable polymers, polyphosphazenes and poly(phosphate esters) or PCL and/or other polymers/copolymer combinations, polyhydroxyalkanonate (PHA) class of polymers, poly(imino carbonates) or Poly(cyanoacrylates).

PCL is a synthetic a-polyester exhibiting a low Tg of around 60° C. which imparts a rubbery characteristic to the material. PCL like other members of this family of polymers such as PLA and PLG, undergoes auto-catalysed bulk hydrolysis). The rubbery characteristics of PCL results in high permeability which may be exploited for delivery of low molecular weight drugs such as steroids and vaccines. PCL is a degradable biopolymer that typically takes more than 1 year to degrade in vivo. However, the semi-crystalline nature of the PCL polymer extends its resorption time to over 2 years.

It is thought to degrade in vivo by hydrolysis to caproic acid and its oligimers and by enzymatic action. In vivo studies have shown that PGA & PLGA fibre scaffolds collapsed after 3 or 4 weeks; hence, in order to improve the integrity of the construct, the addition of PCL to PLGA could stabilise the fibres for long term drug release. PCL may be copolymerized and blended with PLA and PLGA in order to accelerate the degradation.

The delivery device of the present invention can deliver small molecules, or recombinant proteins (for example, chemotherapy drugs) or anaesthetics (for example lidocaine). In particular, the drugs which may be delivered by means of the delivery device of the present invention include, but are not limited to, fluorouracil (5-FU), cisplatin, oxaliplatin, carboplatin and warfarin. The present invention may therefore be applied to a range of diseases, in particular, chronic diseases including (but not limited to) cancers, inflammatory or autoimmune disease, transplant rejection, spinal damage, bone disease, Parkinsons, Alzheimers, etc and to long term anaesthetic agents.

The desired drug may be added to the solution of the matrix material (polymer) by either co-dissolution in a common solvent, dispersion of finely pulverised solid material or emulsification of an aqueous solution of the drug immiscible with the matrix material solution. Dispersion of the solid or dissolved bioactive material in the matrix-containing solution may be achieved by ultrasonication, impeller or static mixing, etc.

The present invention also extends to the use of a drug delivery device (for example a hollow fibre or a microsphere) to deliver drugs to treat chronic diseases in mammals.

The invention may be put into practice in a number of ways and a number of embodiments are shown here by way of example with reference to the following figures, in which:

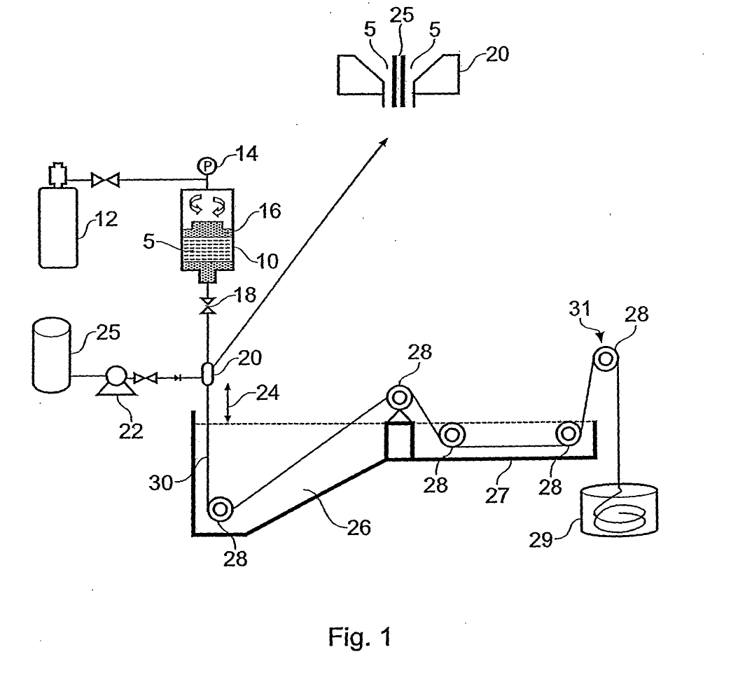

FIG. 1 shows in schematic form the apparatus for the generic spinning procedure for producing a hollow fibre according to the present invention;

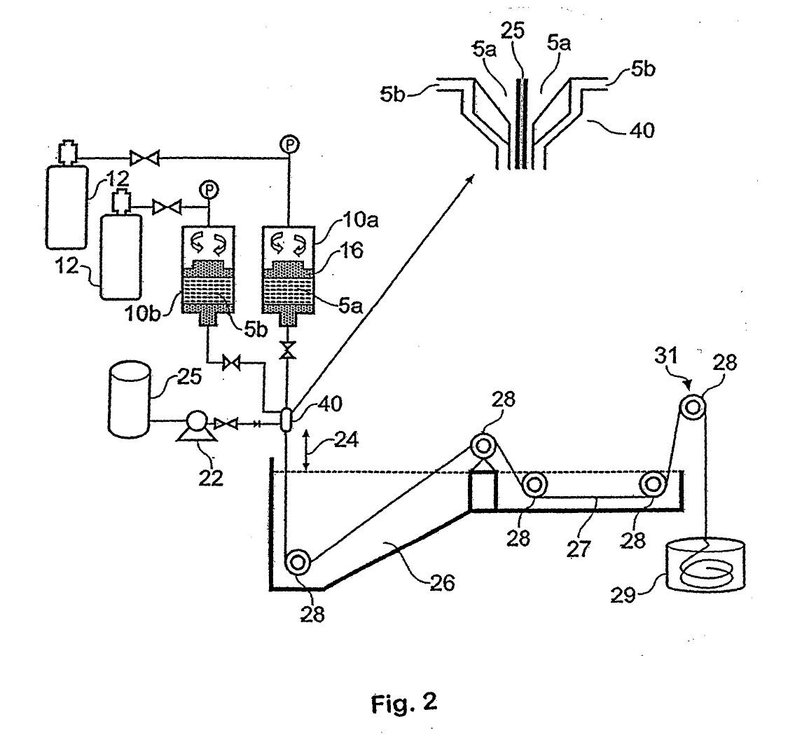

FIG. 2 shows in schematic form the apparatus for the generic spinning procedure for a double layer fibre according to another aspect of the present invention;

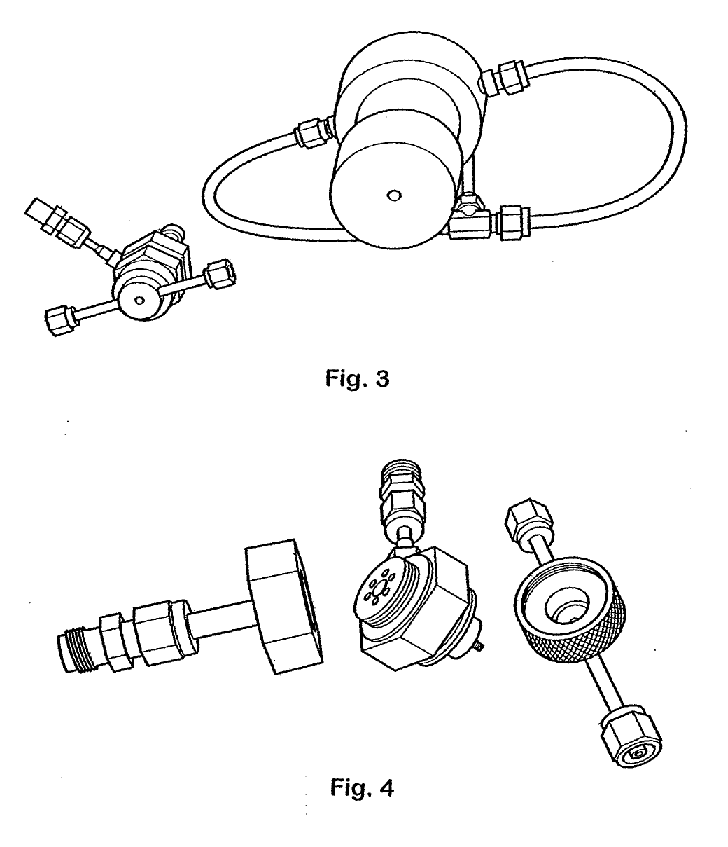

FIGS. 3 to 5 are line drawings of photographs of embodiments of double, triple and quadruple orifice spinnerets according to an aspect of the present invention;

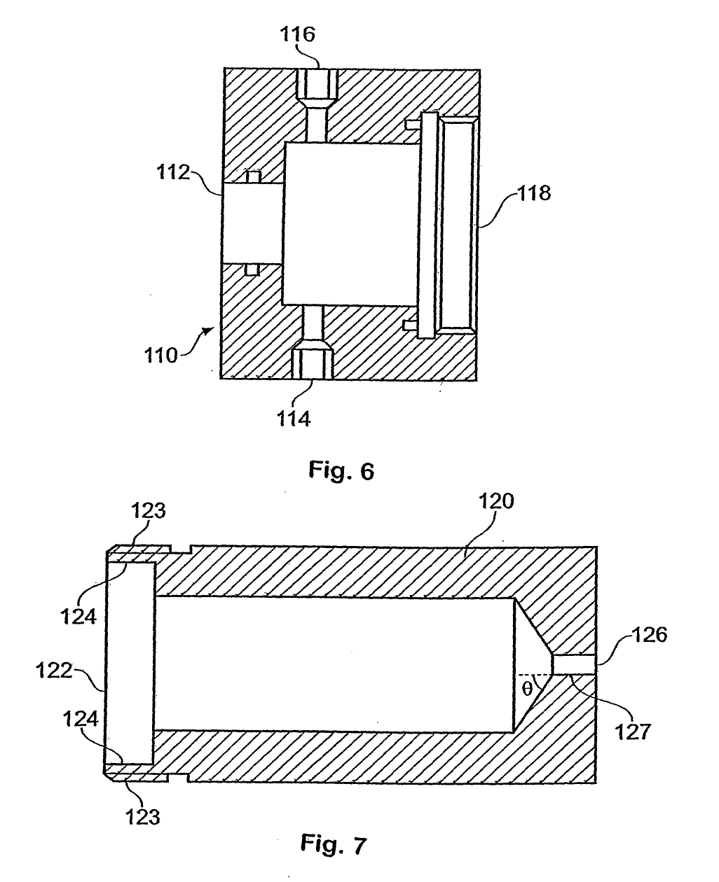

FIGS. 6 to 9 show the component parts for a triple orifice spinneret for use in the production of a double layer fibre;

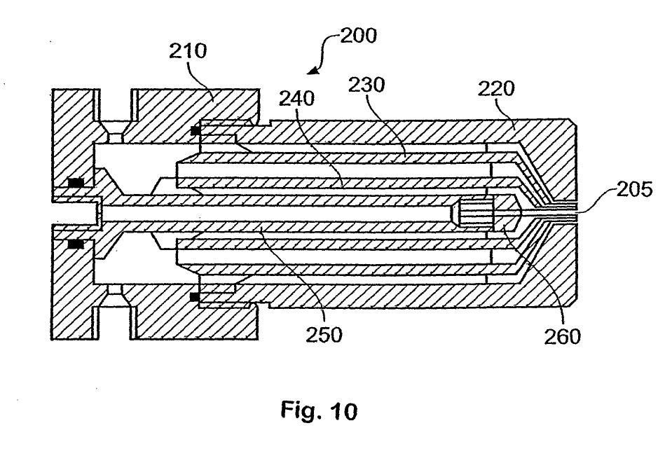

FIGS. 10 to 16 show a quadruple orifice spinneret for use in the production of a triple layer fibre;

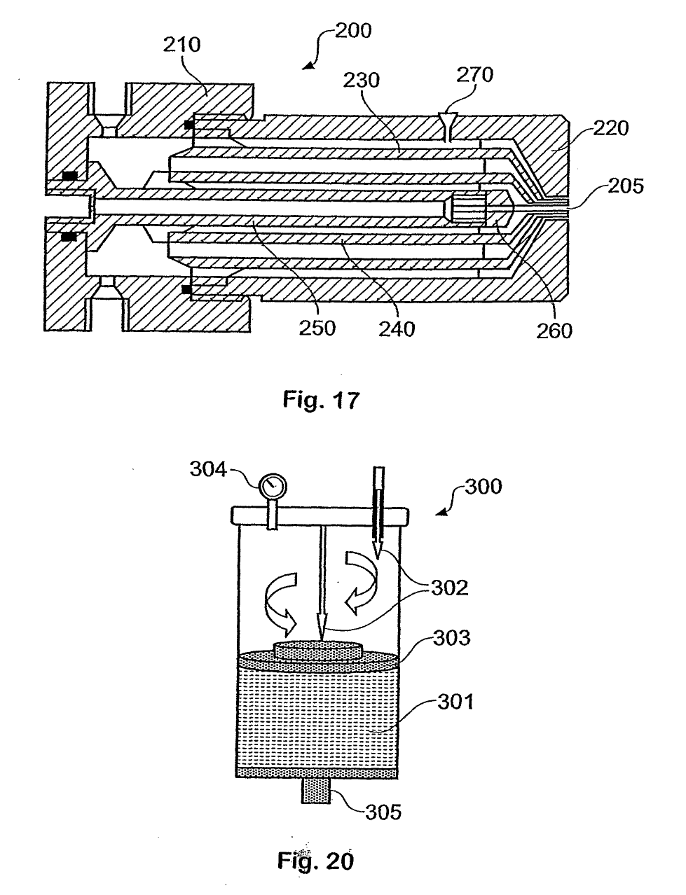

FIG. 17 shows a quadruple orifice spinneret including an additional access point for the introduction of a thin layer of adsorbent as an outer coating;

FIG. 18 shows an alternative design for the quadruple orifice spinneret in which each of the chambers has independent feeds;

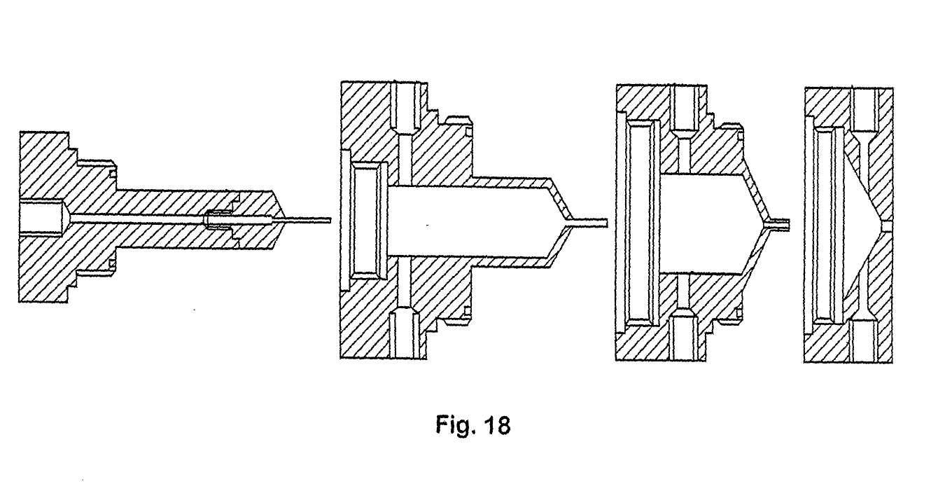

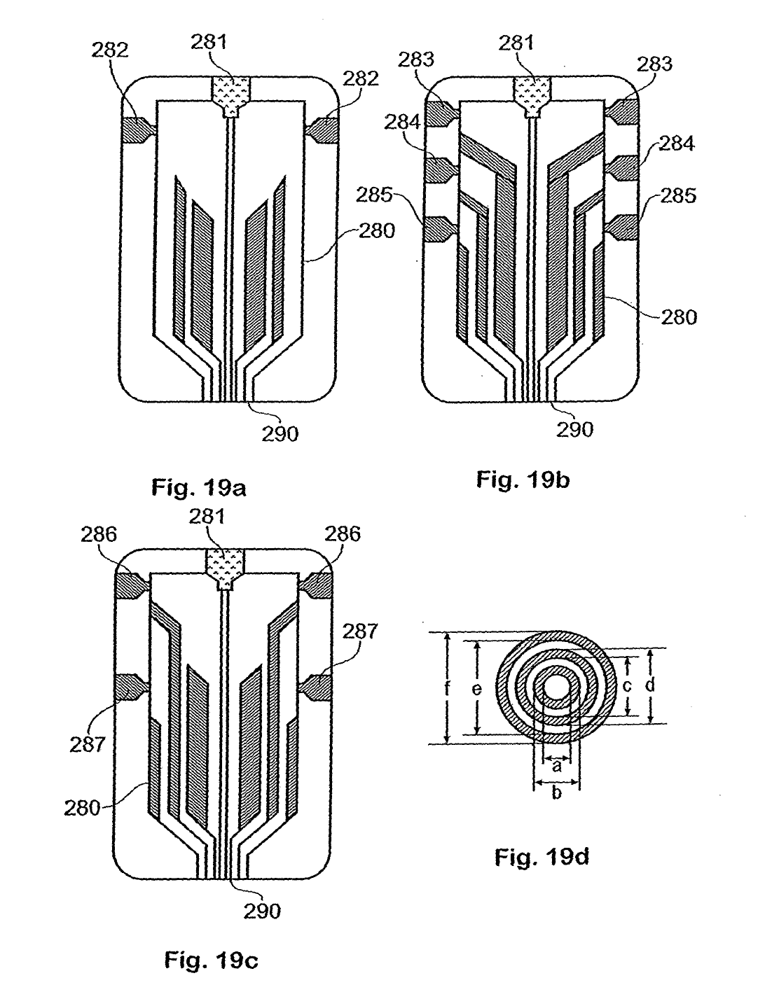

FIG. 19 shows schematically three different designs for producing triple layer fibres using a quadruple orifice spinneret;

FIG. 20 shows a pressure vessel suitable as a delivery vessel for any spinneret of the present invention;

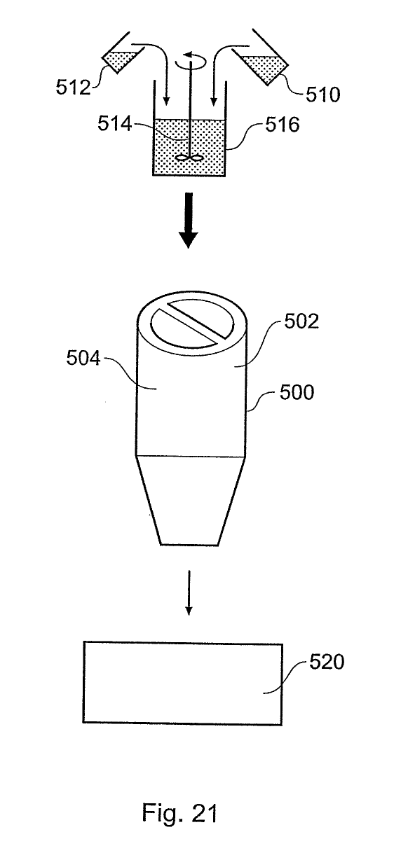

FIG. 21 shows in schematic form, apparatus for the production of microspheres according to the present invention;

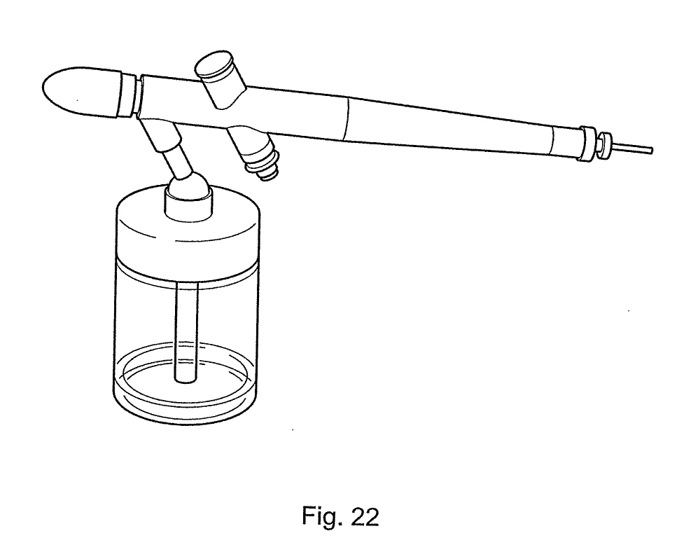

FIG. 22 shows a photograph of a high pressure airbrush which may be used in the production of microspheres according to the present invention.

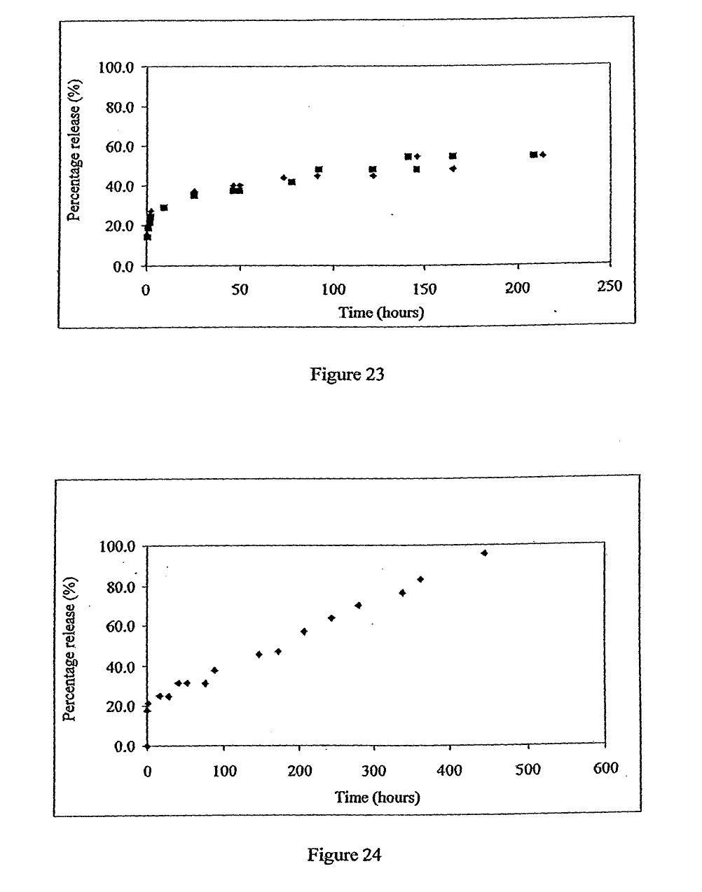

FIG. 23 shows the percentage release of Lidocaine from PLA for samples 1 and 2;

FIG. 24 shows the percentage release of Lidocaine from sample 3;

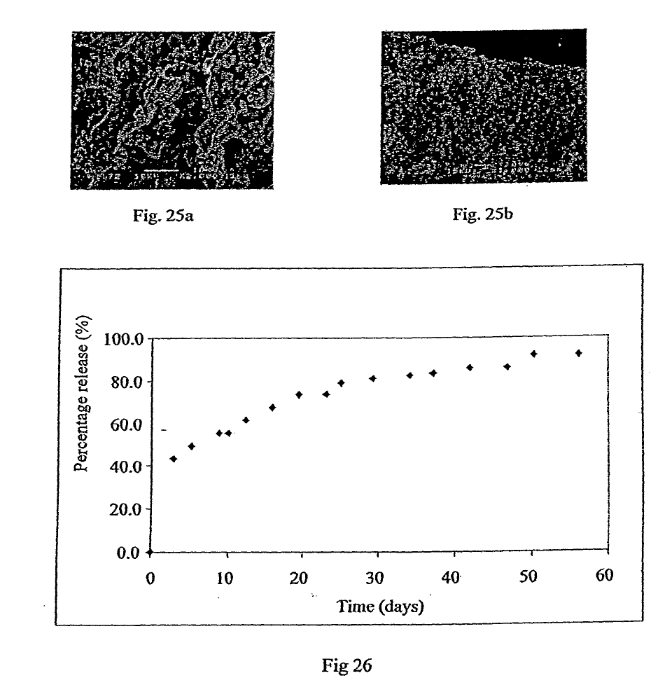

FIGS. 25a and b are SEMs of 25:75 PLGA with Lidocaine in the wall;

FIG. 26 shows the percentage release of Lidocaine and Lidocaine HCL from sample 3;

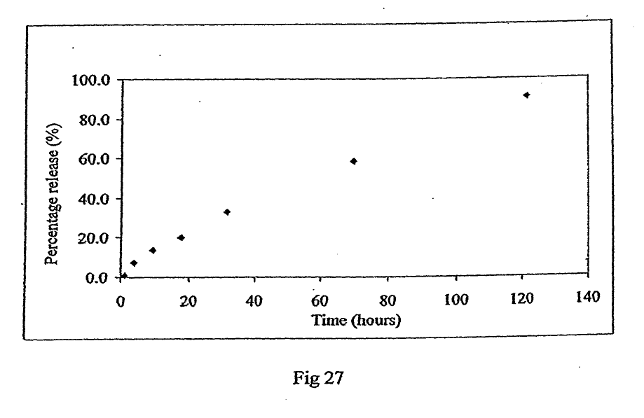

FIG. 27 shows the percentage release of Lidocaine from sample 4;

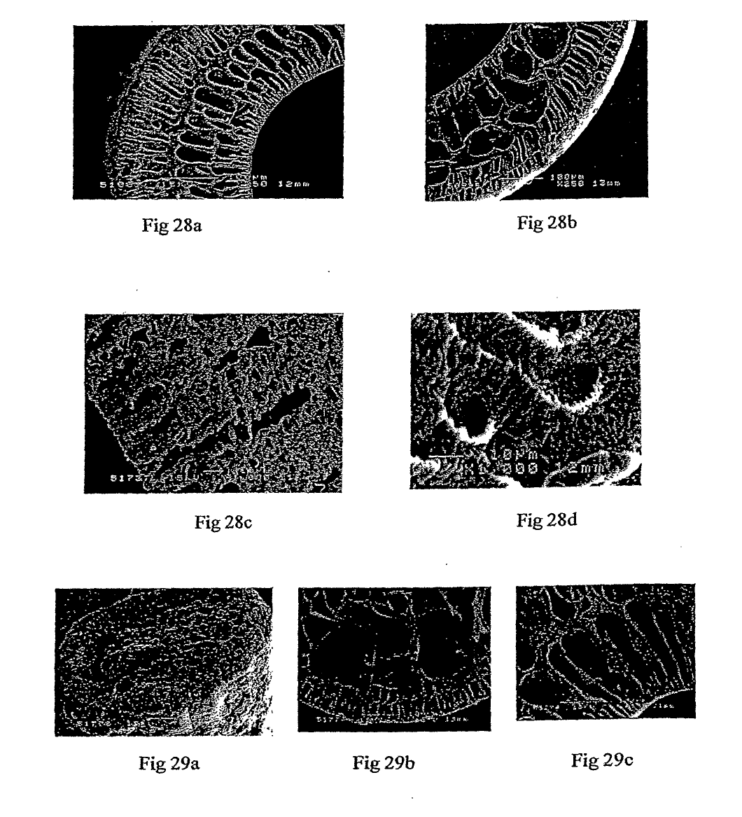

FIGS. 28a-d show SEM micrographs of different samples of hollow fibres before 5-fluorocil injection and before degradation;

FIG. 29a-c shows SEM micrograph of samples after 6 weeks of degradation;

FIG. 30 shows the release of 5-FU from different compositions hollow fibres;

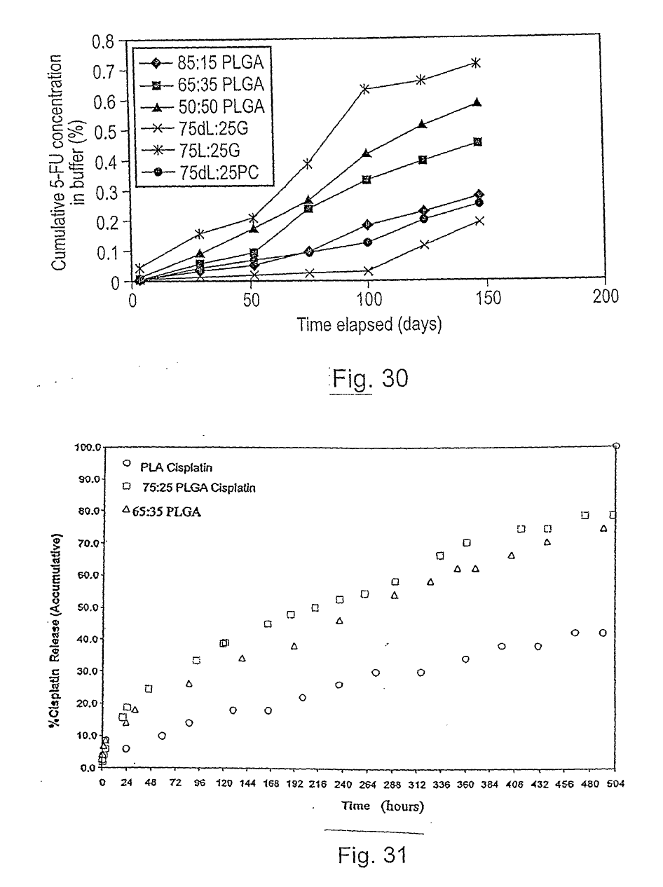

FIG. 31 shows the release of Cisplatin from a range of compositions of hollow fibres;

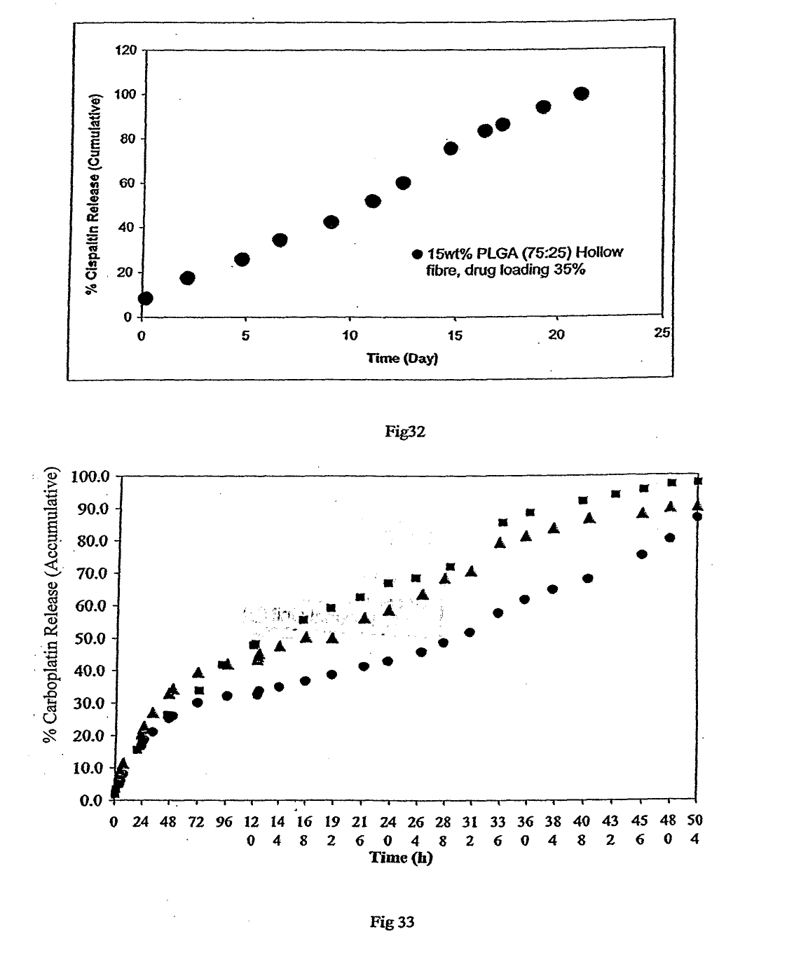

FIG. 32 shows the release kinetics for cisplatin from a PLGA hollow fibre; and

FIG. 33 shows the cumulative percentage mass of carboplatin released from PLGA fabricated fibres over a three week period.

An aspect of the present invention is directed towards a method of production of hollow fibres. This method may generically be described as follows.

### Generic Method for Production of Hollow Fibres

One or more spinning dopes are prepared depending on whether the fibre is to be a single, double, triple, etc layer fibre. For each spinning dope, a suitable solvent is poured into a 500 ml wide-neck bottle, and the desired quantity of polymer is slowly added. The mixture is stirred on a roller to form a polymer solution and once the polymer solution becomes clear, the desired amount of the finely divided powdered drugs to be delivered are slowly added or the drug is dissolved in a suitable solvent first and the drug concentrate is added to the mixture. The mixture is then stirred with an IKA® WERKE stirrer at a speed of 500-1000 rpm for 1-2 days until the drugs are dispersed uniformly in the polymer solution and the mixture is sonicated for 0.5 h in order to obtain an homogeneous mixture, and from the vigorous stirring the mixture is turned into a viscous solution or a gel. The mixture is slightly heated, then filtered through a 100 μm Nylon filter-bag in order to remove any agglomerated or large particles and the mixture is then placed on a rotary pump for 1-2 days to degas and to form a uniform spinning dope.

The fibres are then produced by spinning using an appropriate spinneret which may be followed by slight heat treatment (the temperature is generally kept below 30° C.). Referring to FIG. 1, the mixture 5 is transferred to a stainless steel piston delivery vessel and degassed using a vacuum pump (optionally at a slightly raised temperature) for two hours at room temperature—this ensures that gas bubbles are removed from the viscous polymer dope. The spinning process is then carried out with the following parameters:
1. The heated tank 10 (heating wire around the tank) is pressurised to 2-4 bar using a nitrogen jet 12 and this is monitored by means of a pressure gauge 14. Release of the dope mixture 5 to the spinneret 20 is controlled by means of a piston 16 and valve 18. The delivery vessel is long and small in diameter to maintain uniform pressure for longer periods with in the vessel. The higher the pressure in the tank, and therefore the pressure of the precursor dope passing through the spinneret, the smaller the fibre produced
2. A tube-in-orifice spinneret 20 is used with an orifice diameter of, for example, 2 mm and an inner tube diameter of 0.72 mm, in order to obtain hollow fibre precursors. This double orifice spinneret is for a single layer fibre. For two or more layers, triple or quadruple spinnerets are used and feeds are arranged appropriately. Bore liquid (or the internal coagulant) 25 is also fed to the spinneret 20 and is controlled by means of a gear pump 22. If less bore liquid is pumped through the spinneret the hollow core of the fibre will be smaller and the walls will be thicker. By changing the delivery pressure for each feed the properties of the fibres may be changed
3. The air gap 24 between the bottom of the spinneret 20 and the top surface of the coagulation bath 26 is typically varied in the range 0-3 cm. Increasing the air gap will cause the outer “skin” of the fibre produced to be more dense whereas a smaller air gap will produce a product with more open layers and the fibre will be more porous.
4. The fibre 30 (once extruded from the spinneret) is passed over a series of rollers 28 through a washing bath 27 to a fibre storage tank 29.
5. Water is used as the internal and external coagulator as both bore liquid 25 and as bath liquid in water baths 26 and 27. A low concentration of other solvents also could be added to improve precipitation rate e.g. ethanol, ethyl acetate, acetone.

The precursor is run through the water bath 26 to complete the solidification process and then the hollow fibre 30 is washed thoroughly in the second water bath 27. Care must be taken to ensure that the hollow fibre is not subject to mechanical dragging during the spinning process. Continuity in the pressure is important to deliver polymer dope gel as well as uniform delivery of the internal coagulant in order to avoid entrapment of air and separation of the fibre which would otherwise result in unsuccessful spinning. A guide motor 31 helps to control the movement of the fibre through the water baths. The hollow fibre precursors are then left to soak for 3-4 days in fresh water in the fibre storage tank 29 in order to remove any residual solvent. The precursors are then dried in ambient conditions for seven days.

Apparatus as set out in FIG. 2 may be used to form double layer fibres. The apparatus includes a triple orifice spinneret and two solution feeds. Typical dimensions of the triple orifice spinneret are external layer (dₒᵤₜ 4.0 mm, dᵢₙ 3.0 mm), internal layer (dₒᵤₜ 2.0 mm, dᵢₙ 1.2 mm), and bore (dₒᵤₜ 0.8 mm). Triple and quadruple orifice spinnerets for use in the production of double or triple layer fibres are described in further detail below. For a triple layer fibre, apparatus similar to that shown in FIG. 2 is used but there will be an additional third solution feed for the third layer.

For spinning, two delivery vessels 10a, 10b (or more as may be required) are prepared, one may be pressurised to 2 bar using nitrogen 12, and the other delivery vessel may be further pressurised to 2.5-4 bar using a nitrogen jet. In order to maintain uniform pressures two piston delivery vessel pressure controllers were used. These provide gel feeds 5a and 5b to the triple orifice spinneret with the feed 5a providing the inner layer of the fibre and feed 5b providing the outer layer.

The fibres produced by this method may have two or more layers. This method has the advantage of reduced production costs when compared to prior art methods and also enables the introduction of layers with different functional properties and mixed matrix compositions.

### Triple Orifice and Quadruple Orifice Spinneret

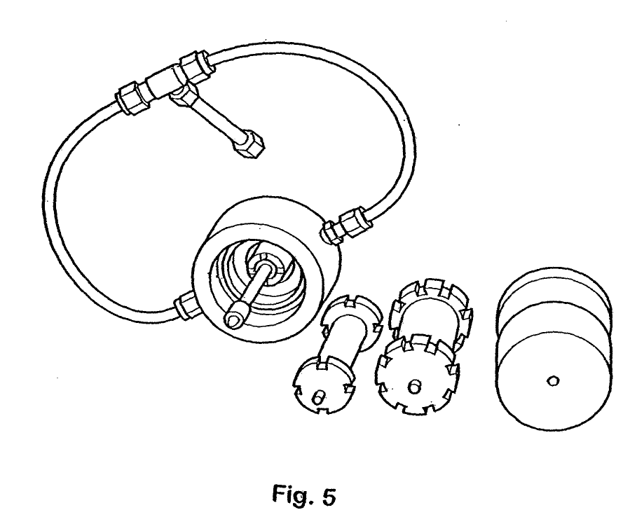

FIG. 3 is a photograph of embodiments of a triple orifice spinneret (left) and a quadruple orifice spinneret. These will be described in further detail below. FIG. 4 is a photograph of the components of one embodiment of a triple orifice spinneret and FIG. 5 is a photograph of one embodiment of a quadruple orifice spinneret.

### Triple Orifice Spinneret

FIGS. 6 to 9 show the components for one embodiment of a triple orifice (double layer) spinneret. FIG. 6 shows a base module 110 to which the precursor feeds are fed and to which the delivery chambers are attached. The precursor feeds may be the same or different and may therefore be fed from the same reservoir (not shown). Alternatively, they may be of different composition and accordingly supplied from different reservoirs under controlled pressure conditions. Feed 112 is for the bore liquid which passes through the centre of the fibre to form the hollow core. Precursor feeds 114, 116 are for the two layers of the fibre. At the outlet 118 of the base module 110 is a screw thread (not shown) to which the delivery chambers are secured.

FIG. 7 shows the outer delivery chamber 120 which controls the precursor feed for the outer layer of the fibre. At the inlet end 122 of the chamber there is provided an external thread 123 to secure the chamber to the base module 110, and an internal thread 124 to which the second chamber 130 is secured. At the outlet, there is a circular orifice 126 at the end of a neck region 127. This orifice 126 will, when the spinneret is assembled, have further outlets passing though it leaving an annular passage through which the material for the outer layer will pass. The outer diameter of this orifice may, for example, be 4 mm. The angle θ of the slope directing the material to the orifice is preferably 60° but may be from 45-65°. Ideally the angles throughout the spinneret should remain constant for all chambers to maintain uniform delivery of the precursor material.

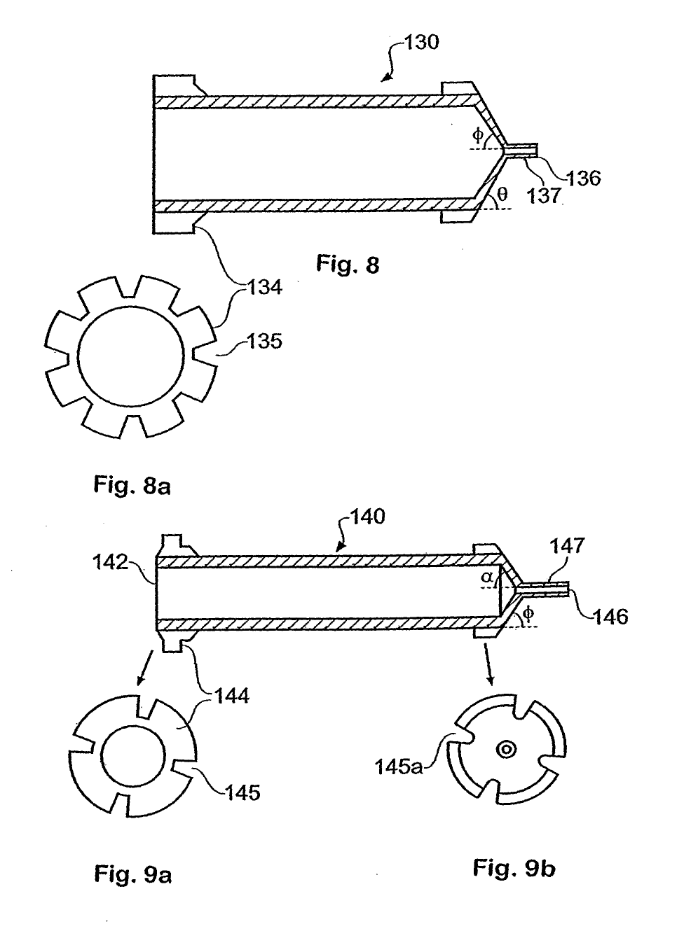

FIGS. 8 and 8a show the second delivery chamber 130 which together with the outer delivery chamber controls the precursor feed for the outer layer of the fibre. At the inlet end 132 of the chamber there is provided a securing ring 134 which has an external thread dimensioned to cooperate with the internal thread 224 of the first chamber 120. The ring 134 has channels 135 cut in the ring at regularly spaced intervals. In a preferred embodiment there are eight channels spaced evenly around the circumference of the ring. These channels permit the flow of the precursor feed for the outer layer to pass from the reservoir, through the spinneret to the outlet 136 of the second delivery chamber.

The outlet takes the form of a circular orifice 136 and the orifice extends in a neck 137 dimensioned to fit inside the neck 127 of the first delivery chamber thereby forming the channel for the intermediate material. This orifice will, when the spinneret is assembled, have further outlets passing though it thereby leaving an annular passage through which the material for the inner layer will pass. The outer diameter of this orifice 137 may, for example, be 3.9 mm and the internal diameter may be 3.5 mm. The angle θ of the external slope of delivery chamber 130 must be the same as θ in the first delivery chamber to maintain the width of the passage through which the outer layer flows. This will also minimise pressure losses in the spinneret. The angle φ of the internal slope which will direct the inner layer of material to the outlet is preferably the same as θ, namely preferably 60°, but may be from 45-65°. As mentioned above, the angles preferably remain constant throughout the spinneret to ensure uniform flow.

FIGS. 9, 9a and 9b show the third delivery chamber 140 which controls the precursor feed for the inner layer of the fibre. At the inlet end 142 of the chamber there is provided a ring 144 which rests against the ring 134 of the second delivery chamber 130. The ring 144 has channels 145 cut in the ring at regularly spaced intervals. In a preferred embodiment there are four channels spaced evenly around the circumference of the ring. These channels permit the flow of the precursor feed for the inner layer to pass from the source, through the spinneret to the outlet 146 of the third delivery chamber. Corresponding channels 145a are also found on the cap at the front end of the chamber which includes the outlet 146.

Again, the outlet takes the form of a circular orifice 146 and the orifice extends in a neck 147 dimensioned to fit inside the neck 137 of the second delivery chamber thereby forming the channel for the intermediate material. The outer diameter of this orifice 147 may, for example, be 2.5 mm and the internal diameter (i.e. the diameter of the hollow core of the produced fibre) may be 2.1 mm. The angle φ of the external slope of delivery chamber 140 must be the same as φ in the second delivery chamber to maintain the width of the passage through which the inner layer flows. This will also minimise pressure losses in the spinneret. The angle α of the internal slope which will direct the bore fluid to the outlet of the spinneret is preferably the same as θ and φ, namely preferably 60°, but may be from 45-65°. Constant angles throughout the spinneret enable uniform delivery of precursor.

The precursor for the inner layer of the fibre passes on the outside of the third delivery chamber, bounded on the other side by the second delivery chamber. The bore liquid passes through the centre of the third delivery chamber to the needle outlet 146.

### Quadruple Orifice Spinneret

FIGS. 10 to 16 show the components for one embodiment of a quadruple orifice, triple layer fibre spinneret. FIG. 10 shows the spinneret 200 assembled. It comprises six members each of which is shown in greater detail in the following figures. Typical dimensions of the spinneret are 140 mm length by 70 mm diameter.

FIG. 11 shows the base module 210 to which the precursor feeds are fed and to which the delivery chambers are attached. The precursor is fed through three feed inlets spaced around the perimeter of the module 210. Two of these inlets are shown as 214, 216. The third (not shown) may be arranged such that it extends out in an orthogonal direction from feeds 214, 216. The feeds may all be the same composition thereby producing a fibre of one composition, but greater strength and with fewer defects, and in this case the inlets are fed from the same reservoir (not shown). Alternatively, the feeds may be of two or three different compositions and accordingly supplied from different reservoirs (not shown) under controlled pressure conditions. Feed 212 is for the bore liquid feed which passes through the precursor material and forms the hollow core in the finished product. At the outlet 218 of the base module 210 is a screw thread (not shown) to which the delivery chambers are secured.

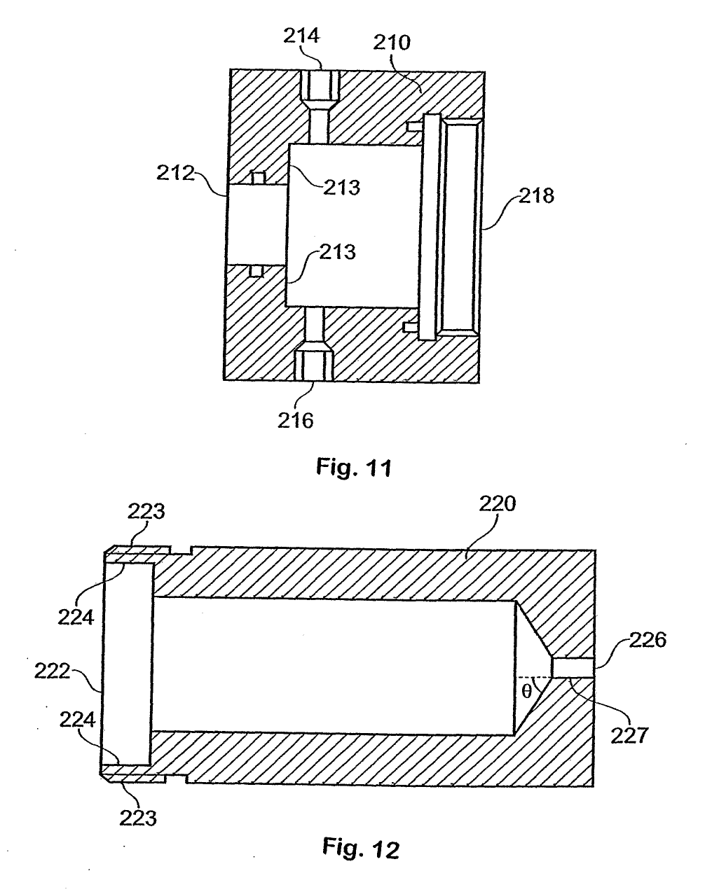

FIG. 12 shows the outer delivery chamber 220 which controls the precursor feed for the outer layer of the fibre. At the inlet end 222 of the chamber there is provided an external thread 223 to secure the chamber to the base module 210, and an internal ridge 224 to support the second chamber 230. At the outlet, there is a circular orifice 226 at the end of a neck region 227. This orifice 226 will, when the spinneret is assembled, have further outlets passing though it leaving an annular passage through which the material for the outer layer will pass. The outer diameter of this orifice may, for example, be 4 mm. The angle θ of the slope directing the material to the orifice is preferably 60° but may be from 45-65°. Ideally the angles throughout the spinneret should remain constant for all chambers to maintain uniform delivery of the precursor material.

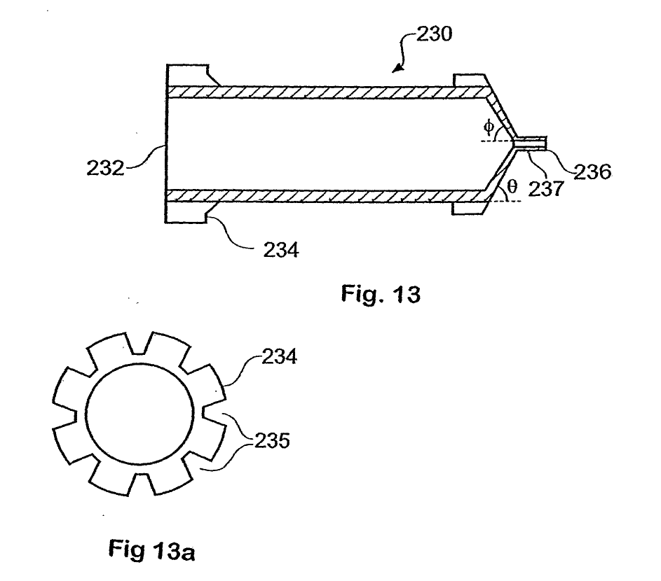

FIGS. 13 and 13a show the second delivery chamber 230 which controls the precursor feed for the intermediate layer of the fibre. At the inlet end 232 of the chamber there is provided a securing ring 234 which has an external thread dimensioned to cooperate with the internal thread 224 of the first chamber 220. The ring 234 has channels 235 cut in the ring at regularly spaced intervals. In a preferred embodiment there are 8 channels spaced around the circumference of the ring. These channels permit the flow of the precursor feed for the intermediate layer to pass from the source, through the spinneret to the outlet 236 of the second delivery chamber.

Again, the outlet takes the form of a circular orifice 236 and the orifice extends in a neck 237 dimensioned to fit inside the neck 227 of the first delivery chamber thereby forming the channel for the intermediate material. Again, this orifice will, when the spinneret is assembled, have further outlets passing through it thereby leaving an annular passage through which the material for the inner layer will pass. The outer diameter of this orifice 237 may, for example, be 3.9 mm and the internal diameter may be 3.5 mm. The angle θ of the external slope of delivery chamber 230 must be the same as θ in the first delivery chamber to maintain the width of the passage through which the intermediate layer flows. This will also minimise pressure losses in the spinneret. The angle φ of the internal slope which will direct the inner layer of material to the outlet 205 is preferably the same as θ, namely preferably 60°, but may be from 45-65°. As mentioned above, the angles preferably remain constant throughout the spinneret to ensure uniform flow.

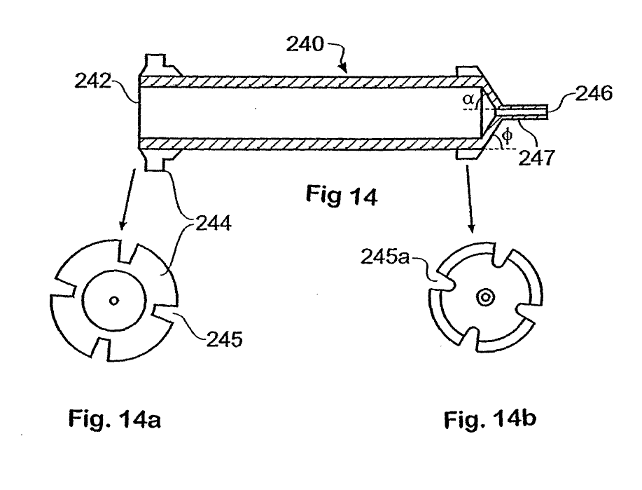

FIGS. 14, 14a and 14b show the third delivery chamber 240 which controls the precursor feed for the inner layer of the fibre. At the inlet end 242 of the chamber there is provided a ring 244 which rests against the ring 234 of the second delivery chamber 230. The ring 244 has channels 245 cut in the ring at regularly spaced intervals. In a preferred embodiment there are four channels spaced around the circumference of the ring. These channels permit the flow of the precursor feed for the intermediate layer to pass from the source, through the spinneret to the outlet 246 of the third delivery chamber. Corresponding channels 245a are also found on the cap at the front end of the chamber which includes the outlet 246.

Again, the outlet takes the form of a circular orifice 246 and the orifice extends in a neck 247 dimensioned to fit inside the neck 237 of the second delivery chamber thereby forming the channel for the intermediate material. Again, this orifice will, when the spinneret is assembled, have further outlets passing though it thereby leaving an annular passage through which the material which will form hollow core of the fibre will pass. The outer diameter of this orifice 247 may, for example, be 2.5 mm and the internal diameter may be 2.1 mm. The angle φ of the external slope of delivery chamber 240 must be the same as φ in the second delivery chamber to maintain the width of the passage through which the inner layer flows. This will also minimise pressure losses in the spinneret. The angle α of the internal slope which will direct the bore fluid to the outlet 205 is preferably the same as θ and φ, namely preferably 60°, but may be from 45-65°. Constant angles throughout the spinneret enable uniform delivery of precursor.

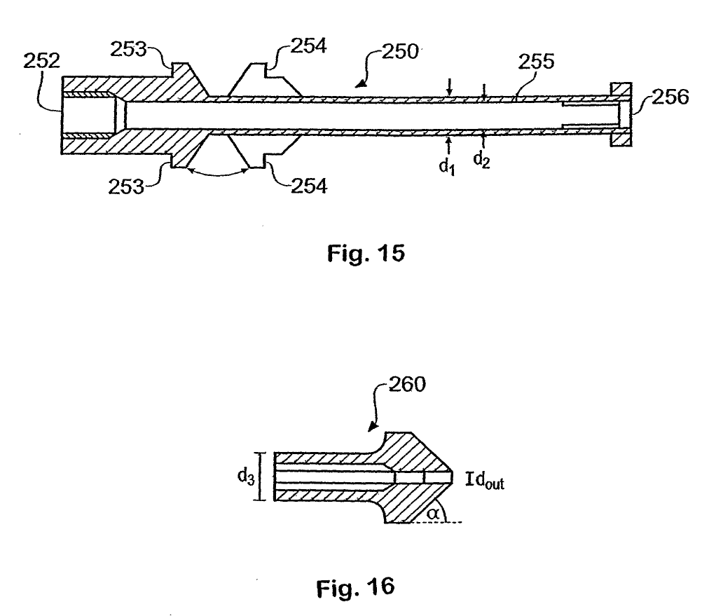

FIG. 15 shows a further chamber 250 through which the bore fluid flows. Attached to this chamber at the front end is a bore needle 260 as shown in enlarged form in FIG. 16. The bore needle 260 will define the dimension of the inner hollow core of the fibre and may therefore be varied from embodiment to embodiment as appropriate. The inlet 252 of the chamber 250 is arranged to cooperate with the bore liquid inlet feed 212 of base module 210. The shoulders 253 abut the inner surface 213 of the base module 210. The shoulders 254 abut the ring 244 at the inlet end of third chamber 240. The shoulder portion 254 has matching channels which line up with the channels 245 in ring 244. There is also a small gap below the shoulder 254 to allow further passage of the precursor fluid. The core 255 of the chamber 250 has an external diameter d₁ and an internal diameter d₂. Preferred values for d₁ and d₂ may be 8 mm and 4 mm respectively but any values in the range 1-20 mm may be appropriate for a specific embodiment.

At the front end of chamber 250 there is an outlet 256. Towards this end the core may increase in internal diameter to accommodate the bore needle 260 (see FIG. 16). For example the internal diameter may increase from 4 mm to 5.2 mm. The bore needle 260 is arranged to fit inside the outlet 256 of chamber 250 as a snug push fitting. The dimension d₃ of the bore needle may, for example, be 5 mm to fit inside the outlet end 256 of the chamber 250 having an internal diameter of 5.2 mm. The diameter of the needle dₒᵤₜ may be in the range 0.1-5 mm, more preferably 0.5-3 mm, for example 1 mm. This defines the size of the hollow core of the fibre. The angle α should be the same as in the third delivery chamber 240 to maintain the width of the passage through which the precursor fluid flows. As indicated above, α is preferably 60°, but may be in the range from 45-65°.

FIG. 17 shows a similar view to FIG. 10, but the spinneret has an additional access point 270 for the introduction of a thin layer of adsorbent or other functional material. This will form an outer coating in addition to the three layers of the fibre. This thin layer may be present to help the selectivity of the fibre for a particular adsorbate.

FIG. 18 shows an alternative design for the quadruple orifice spinneret in which each of the chambers has independent feeds. The arrangement of the chambers is similar to that described with respect to FIGS. 10 to 16 above but each chamber has clear and separate precursor feeds which do not all pass through the base module.

FIG. 19 shows schematically three different designs for producing triple layer fibres using the quadruple orifice spinneret. FIG. 19a shows a spinneret 280 for the delivery of gel precursor of one composition from a single piston pressure vessel 282. The fibre produced is stronger and defect free. The bore liquid passes from reservoir 281 through the centre of the spinneret 280 to form the hollow core of the fibre. Each of the channels leads to the outlet 290 which may take the form shown in FIG. 19d. FIG. 19b shows a spinneret 280 for the delivery of three different compositions from three different delivery vessels 283, 284, 285, the pressure of which is controlled independently. Each composition may have different types of adsorbent with different functional properties attached to them. Using this system it is possible to produce compact fibres with very small particles to achieve small pores of the order of 1-2 nm.

FIG. 19c shows a design for the delivery of two different compositions. The two inner layers are of the same composition fed from delivery vessel 286 and the outer layer is of a different composition from delivery vessel 287. This fibre has the advantage of a stronger fibre with fewer defects of a first composition, with an outer layer which is specifically chosen to have the functional properties required, for example in the choice of adsorbent.

FIG. 19d shows a typical arrangement of the outlet 290 of the spinneret 280. The three concentric rings of precursor each have a thickness of 0.5 mm. For example, the inner core formed by the bore liquid may have a diameter a of 1.1 mm. The outer diameter of the first layer of precursor then has a diameter b of 2.1 mm. The intermediate layer has an inner diameter c of 2.5 mm and an outer diameter d of 3.5 mm. The outer layer has an inner diameter of 3.9 mm and an outer diameter of 4.9 mm.

FIG. 20 shows a pressure vessel 300 suitable as a delivery vessel to the spinneret. The precursor gel 301 is maintained under pressure by means of jets of nitrogen 302 being applied to a plate type piston 303. The pressure is measured by means of a pressure gauge 304. The precursor is fed out of the vessel 300 through the outlet means 305 to the appropriate feed of the spinneret. As shown in FIG. 20, the outlet is conically shaped and may be, for example, 5-15 mm in diameter, preferably 10 mm. The vessel 300 is made of stainless steel and may have dimensions of 150-200 mm height by 60-80 mm diameter. The delivery vessel can also be heated by a heating tape.

Referring to FIG. 21, an apparatus suitable for producing microspheres according to the present invention is shown schematically. A biocompatible polymer is dissolved in a suitable solvent 510, and a finely divided drug 512 is dissolved or dispersed in the solvent containing the polymer material and the materials are mixed by a mixer 514 in a beaker 516. The mixture is placed in an airbrush jar and is then sprayed via line 502 into an antistatic chamber 500. Simultaneously, water saturated air 504 is introduced into the chamber to achieve solidification of the beads. The two sprays are introduced into the antistatic chamber at an angle to achieve a cyclonic action to ensure good contact between the sprays.

The mixture has a substantial residence time in the chamber (for example 2-10 minutes) to allow the droplets formed to undergo phase inversion. The two sprays are fed into the chamber in the same direction so that they are co-current. The beads are formed into microspheres by extraction of the solvent. This may be by any suitable means, for example heating or vacuum treatment. The collected microspheres are then dried 520 to reduce the level of solvent still further to 0-2% by weight.

FIG. 22 shows an example of an airbrush jar which may be used in this method or the bath method or producing beads (not shown). The airbrush jar has a very small nozzle to enable the production of small droplets (10-50 μm). The mixture is generally sprayed at a high pressure (3-3.5 bar) using compressed air. The size of the beads is controlled by both the pressure of the spray and the viscosity of the polymer dope.

In the bath method, the mixture of solvent, polymer and drug is sprayed directly into a water bath and not into an antistatic chamber. Water-solvent demixing occurs and drug encapsulated beads are produced. The microspheres produced by both methods were found to be of a uniform size, the volume of which is determined by the spraying pressure. Generally the spheres produced by the bath method have a smaller pore size and have a more dense outer skin.

### EXAMPLES

In the following examples, the following abbreviations may be used for the different chemicals used.
DCM—Dichloro-methane
EA—Ethyl acetate
PBS—Phosphate buffer saline (adjusted to pH 7.4 with 0.1M NaOH)
PVA—Poly-vinyl-alcohol
PLA—Poly(L-lactic) acid
PLGA—Poly(lactic-co-glycolic) acid (75:25 and 65:35)

### Experimental Method

### Spinning of Hollow Fibre

### Materials

PLA and PLGA (16-30 wt %) were dissolved in DCM, acetone or ethyl-acetate solvent. Finely divided Lidocaine powder or Cisplatin was sieved and a known amount was added to the polymer solution. The internal and external coagulants are distilled water and tap water respectively.

### Preparation of Polymer Dope

Spinning solution was prepared by dissolving a specific amount of polymer to DCM, acetone or ethyl-acetate solvent. The polymer was then allowed to dissolve completely while being mixed on a roller mixer.

### Hollow Fibre Spinning

The polymer solution was degassed in a vacuum chamber to completely remove gas bubbles and then transferred to the piston delivery vessel (FIG. 1). The delivery vessel was then sealed and pressurised to 2-4 bar using nitrogen gas. The solution was extruded through a tube-in-orifice spinneret with 1 mm orifice diameter and 0.3 mm inner tube diameter. Water was used as an internal coagulant to form a hollow fibre and the flowrate pressure and other parameters were altered to produce fibres with ranges of properties (eg diameters, porosity and wall thickness).

The hollow fibre was then passed through a series of water baths to aid the phase inversion process (as shown in FIG. 1). The air gap between the spinneret and water bath was usually at 0-10 cm. A suitable roller rotation rate needs to be selected to prevent mechanical dragging of the fibre. After spinning, the fibres were left to soak in fresh water for 2-4 days to remove residual solvent. The fibres were then dried at ambient conditions.

### Drug Loading and Encapsulation Efficiency for Lidocaine

Hollow fibres, either newly spun or from post-release experiments, were dissolved in 10-20 mL of DCM. After complete dissolution, a known amount (usually 10 mL) of 0.1M H₂SO₄ solution was added to the solvent to extract the lidocaine (as lidocaine is more soluble in acidic solutions). The UV absorbance (at 262 nm) of the acidic solution was measured and this lidocaine concentration was multiplied with the amount of H₂SO₄ solution used (i.e. dilution factor) in order to give the total amount of lidocaine in fibre.

In order to determine the amount of lidocaine encapsulated in microspheres, approximately 0.1 g of microspheres was weighed and dissolved in 5 mL of DCM followed by 0.5 h sonication. To this solution, 5 mL of 0.1M H₂SO₄ was added. 3 mL of sample solution from the H₂SO₄ solution was withdrawn for lidocaine concentration determination taking into account of the dilution factor. H₂SO₄ solution was used as the blank sample while measuring absorbance of the sample at 262 nm and as a result, the drug loading and encapsulation efficiency could be estimated.

$$Drug Loading ( \% ) = \frac{Total Drug Encapsulated ( mg )}{Total Microsphere Produced ( mg )} \times 100 \%$$

$$or \\ = \frac{Total Drug Encapsulated ( mg )}{Total Weight of Fibre used ( mg )} \times 100 \%$$

$$Encapsulation Efficiency ( \% ) = \frac{Total Drug Encapsulated ( mg )}{\begin{matrix}Theoretical Maximum \\ Drug Used ( mg )\end{matrix} } \times 100 \%$$

The effectiveness of this method was tested by dissolving 9 mg of lidocaine powder in DCM and extracting it into 10 ml of 0.1M H₂SO₄ solution. The absorption reading obtained was 1.6 which in turn gave a lidocaine concentration of 0.93 mg/mL. The corresponding ±2% error may be due to the variation of the instrument and this method was found to be reliable.

### In Vitro Drug Release Analysis

Microspheres weighing between 0.1 and 0.3 g were suspended in a suspension of 5 mL of PBS (pH 7.4) incubated in a shaking water bath (100 rpm) at 37° C. for up to three weeks. Absorbance measurement was taken at different time intervals. I.e. Initially 0.5 h, then hourly and daily etc. In the case of hollow fibres, 20 mL of PBS (pH 7.4) was used.

Selected fibres were cut into equal lengths, weighed and injected with lidocaine HCL solution, before sealing the ends with Araldite epoxy resin. The fibres were then rinsed with distilled water to wash away any drug residue on the surface before immersing into 20 mL of PBS buffer solution (pH 7.4). For release experiments involving only incorporated lidocaine, the fibres were first weighed before being placed into buffer solution. The fibre samples were placed in a shaking water bath (100 rpm). At regular intervals, the concentration of lidocaine in the buffer was measured using an UV-visible spectrophotometer at 262 nm. The buffer was refreshed when the UV absorbance indicated plateau.

A similar procedure was adapted for cisplatin. At regular intervals, the concentration of Cisplatin in the buffer was measured using UV-visible spectrophotometer at 298 nm. The results are presented in FIG. 31.

### Particle Size Analysis (for Beads)

Microparticle size was measured using a laser diffracting analyser, Malvern Mastersizer X. Each sample (suspended in cyclohexane with 1 mg/mL lecithin) was analysed a total of 2000 times to give an average value for the particle diameter or undersize. Briefly, in a laser diffraction particle analyser, a representative ‘ensemble’ or particle passes through a broadened beam of laser light which scatters the incident light onto a lens. This lens focuses the scattered light onto a detector array and using Mie theory (which solves Maxwell's equations exactly for the boundary conditions of a spherical particle) the particle size distribution is inferred from the collected diffracted light data.

### Examples 1-4

### Lidocaine Hollow Fibres

### Spinning of Hollow Fibre Membranes

Hollow fibre membranes were spun with lidocaine powder incorporated into the polymer solution in order to produce fibres with a high drug loading (in the examples, lidocaine loadings were carried out to fulfil the long term anaesthetic requirements). Range of polymers/compositions with varying degradation rates were used.

TABLE 1

| Ex | | Polymer | Lidocaine | |
|---|---|---|---|---|
| No. | Polymer | wt % | wt % | Notes |
| 1. | 100DL-PLA | 20 | 20 | Lidocaine powder added to |
| | | | | solution |
| 2. | 100DL-PLA | 16 | 20 | Lidocaine powder added to |
| | | | | solution |
| 3. | 75:25 PLGA | 20 | 40 | Lidocaine powder added to |
| | | | | solution |
| 4. | 65:35 PLGA | 25 | 40 | Drug pre-dissolved in ethanol |

By altering the concentration, composition, molecular weight of the polymer and the spinning parameters, hollow fibre membranes of varying thicknesses and controlled porosity are produced; the less viscous the solution (lower polymer concentration), the finer the fibres spun. 100DL-PLA fibres were found to be very stiff due to the high modulus of PLA and seem to be suitable for stents.

### Drug Encapsulation Efficiency

The amount of drug entrapped successfully in the walls of the hollow fibre membrane was determined. The effectiveness of this method was tested by dissolving 9 mg of lidocaine powder in DCM and extracting it into 10 mL of 0.1M H₂SO₄ solution. The test was repeated 5 times and the resultant concentration was 0.93 mg a standard error of 0.10 mg/mL.

TABLE 2
Drug loading and encapsulation efficiency
of hollow fibre membranes

| Ex | | Drug Loading | Encapsulation Efficiency |
|---|---|---|---|
| No. | Polymer | (wt %) | (%) |
| 1. | PLA 20% | 4.43 | 22.15 |
| 2. | PLA 16% | 5.12 | 25.60 |
| 3. | 75:25 PLGA | 7.72 | 19.23 |
| 4. | 65:35 PLGA | 6.22 | 15.55 |

### Sample No 1

0.012 g of Ex. 1 was dissolved in DCM and 10 mL of 0.1M H₂SO₄ solution added to the solvent to extract the lidocaine. After vigorous shaking, the UV absorbance (at 262 nm) of the acidic solution was found to be 0.114, which correlated to a concentration of 0.0532 mg/mL. Therefore the total amount of lidocaine present was 0.532 mg. The drug loading of Sample No 1=(0.532 mg Lid/12 mg fibre)×100%=4.43%. The encapsulation efficiency=4.43/20×100%=22.15%

### Sample No 2

0.0326 g of Ex. 2 was dissolved in DCM and 20 mL 0.1M H₂SO₄ solution was added. UV absorbance of the acidic solution was 0.165 indicating a concentration of 0.0835 mg/mL. Therefore there was 1.67 mg of lidocaine present which resulted in a drug loading of 5.12%. Encapsulation efficiency 5.12/20×100%=25.6%

### Sample No 3

0.02 g of Ex. 3 was dissolved and lidocaine extracted into 10 mL of 0.1M H₂SO₄ solution. Absorbance at 262 nm was 0.284 therefore there was 1.543 mg of drug in the solution. Drug loading was calculated as 7.72% and the encapsulation efficiency was 19.23%.

### Sample No 4

0.062 g of Ex. 4 was dissolved and lidocaine extracted into 10 mL of 0.1M H₂SO₄ solution. Absorbance at 262 nm was 0.673 therefore there was 3.856 mg of drug in the solution. Drug loading was calculated as 6.22% and the encapsulation efficiency was 15.55%.

### In Vitro Drug Release Studies

The release of lidocaine into PBS (pH 7.4) was measured by UV-Visible spectrophotometry to determine its in vitro release profile. Lidocaine was incorporated into the walls of hollow fibres Sample 1, Sample 2, Sample 3 and Sample 4 and release was facilitated by diffusion initially, followed by degradation of the polymer. Additionally, lidocaine HCl solution (20 mg/mL) was injected into the lumen of sample 3 with the ends sealed in order to determine the diffusion release of the drug.

At the end of each experiment, the amount of drug remaining in the polymer was calculated by dissolving the hollow fibres in DCM, followed by extraction of the drug into 0.1M H₂SO₄ solution. The release profile results were then represented as a percentage release over time, where:

$$\% Release = \frac{Cumulative amount of drug released}{( Final amount released + Amount remaining )} \times 100 \%$$

Release of Lidocaine from Sample 1 and 2

Lidocaine powder was incorporated into the walls of the fibre (44.3 mg of lidocaine per 1 g of fibre). Two experiments were run concurrently and both their drug release profiles were obtained.

After incubation into PBS (pH 7.4), there was an immediate release of lidocaine into the surrounding medium, with 15% of the total contained drug released within the first 30 minutes (see FIG. 23). By 4 hours, 30% had been released. This was due to the diffusion of non-trapped drug or drug near the surface of the polymer wall, as water diffuses into the polymer matrix to form a homogenous distribution. 24 hours after immersion, a further 10% of drug had diffused out of the hollow fibre. Subsequent release was slow with barely any lidocaine released after the third day.

However, when the residual drug content of the fibres was determined after 17 days into the experiment, it was found that only 45% of the lidocaine had been released with a further 55% still remaining in the hollow fibre wall. Due to the slow degradation of 100-DL PLA, the drug was entrapped within the polymer matrix and there was insufficient degradation during the period of experiment to facilitate the release of the remaining drug confirming that PLA is suitable for long term drug release.

Release of Lidocaine from Sample 3

Lidocaine powder was incorporated into the polymer matrix of sample 3 which was made of 75:25 PLGA. Initial drug release was similar to that of sample-2 as 18% of lidocaine was released in the solution. After 24 hours the total amount released had increased to 21% (see FIG. 24).

This was followed by a slow gradual release (zero order kinetics), reaching 100% release after 20 days post-immersion. This was accompanied with degradation of the fibres into smaller particles therefore the release of lidocaine was diffusion as well as erosion-controlled. Further absorption readings could not be taken accurately due to the presence of polymer particles suspended in the solution. FIGS. 25a and 25b are SEMs of 25:75 PLGA with Lidocaine in the wall.

Release of Lidocaine and Lidocaine HCl from Sample 3

To increase the drug loading of the hollow fibres, lidocaine HCl solution was injected into the hollow fibres in addition to having drug particles in the fibre wall. The resulting release profile showed a very quick release effect with 54% of the total drug loaded released in the first 2 days. The results shows that initially the liquid drug diffused quickly into bulk solution (see FIG. 26).

After 24 days, 72% of lidocaine was released and this was followed by a very gradual release for the next 22 days. As the ends of the fibres were sealed initially, water uptake into the hollow fibre did not proceed as quickly as plainly immersed fibres and therefore degradation was also slower. At the end of 23 days, 80% of the drug had been released.

The injection of solution into the hollow fibre lumen was able to increase the drug loading considerably, as 0.1 g of fibres was able to contain 20 mg of lidocaine.

Release of Lidocaine from Sample No 4

Lidocaine was incorporated into the walls of Sample 4 which was made of 65:35 PLGA, the fastest degrading polymer used in the experiments conducted. The drug powder was first dissolved in ethanol before it was added into the polymer solution to ensure that there was good dispersion of drug. The resulting release profile showed a more gradual release of drug initially; only 7% was released in the first 60 minutes. After one day around 20% of the total drug had diffused out into the PBS solution (pH 7.4) (see FIG. 27).

Subsequent release continued to be gradual while degradation of the polymer took place more rapidly than the previous experiments. Degradation of the polymer was quite extensive leading to a particle suspension in the buffer.

### Development of Fibres for 5-Fluorouracil Tests and Cisplatin Tests

### Materials

The polymers used in the spinning processes were poly(D,L-lactic acid) (PLA) and polycaprolactone (PCL) and PLGA. Dichloromethane, ethyl acetate, 1-methyl-2-pyrrolidone (NMP) (99.5%) was used as solvents to dissolve the PLA, PLGA polymer and prepare the polymer solution. Acetone on the other hand, was used to prepare the PCL polymer solution. Distilled water purified was used as an internal coagulant, as well as the external coagulant. Commercial liquid form 2.5 g/100 ml 5-florouracil (5FU) and cisplatin 10 mg/ml was used for pharmacokinetic studies. Also cisplatin, carboplatin and 5FU powdered form used for fibre and microsphere preparations. For the medium in drug release experiment, phosphate buffer saline (PBS) pH 7.4 was used.

### Spinning Solution Preparation

The required quantity of organic solvent (ethyl acetate, dichloromethane, acetone, or NMP), was poured into a one-litre wide-neck bottle and then the desired quantity of polymer (PLA, PLGA PCL and the mixture of co-polymers) was slowly added. The mixture was stirred on a rotary roller to form the polymer solution. The finely divided powdered drug (eg. cisplatin, carboplatin, 5FU or oxaliplatin and other) was added to the polymer mixture and vigorously stirred to achieve a homogeneous mixture. This ensured that drugs were only exposed to solvents limited time. During the triple layer spinning the drug particles were suspended in a saline solution or in purified water which was injected into the middle layer of the fibre through a syringe pump. This process aids the solidification process entrapping the drugs efficiently within the polymer matrix. For PCL, the polymer solution was needed to be heated in a water bath to 55° C. to improve dissolution of poly s-caprolactone.

TABLE 3
Spinning Parameters

| | |
|---|---|
| Coagulation bath temperature (° C.) | 20 |
| Injection rate of internal coagulant (ml/min) | from 2 to 14 |
| Nitrogen Pressure (bar) | 2-4 |
| Air gap (cm) | 0 and 3 |
| Linear extrusion speed (rpm) | varies |
| Bore liquid | purified water |
| External coagulant | purified water |

### Bio Polymeric Hollow Fibre Spinning

The polymer solution was degassed for 24 hours at room temperature before the spinning process in order to completely get rid of any gas bubbles of viscous polymer solution in the spinning process. The polymer solution was then transferred to a piston delivery vessel. The tank was pressurised to 2-4 bar using nitrogen during the spinning process (see FIG. 1).

A tube-in-orifice spinneret with orifice diameter and inner tube diameter of 2.0/0.72 (mm) was used to produce the hollow fibres. The air gap was kept at 0 cm and 3 cm, and water was used as the internal and external coagulant for all spinning runs. Finally, in forming the hollow fibre, it was passed through a series of water baths to aid the solidification process. The hollow fibre was then washed thoroughly in a second water bath. During spinning care was taken to ensure continuity of the pressure and internal water support in order to avoid entrapment of air and separation of the fibre, which would eventually result in an unsuccessful spinning. The hollow fibres were left to soak in fresh water; this was essential for thorough removal of residual solvent and then vacuum dried to remove any residual solvents. Prepared drug encapsulated fibres were freeze dried/vacuumed packed for characterisation

### Characterisation

### Scanning Electron Microscope (SEM)

The scanning electron microscope (SEM) was used for the characterisation of hollow fibres before drug (5-fluorouracil) injection and after drug injection into lumen of the fibres. The surface structure, particle size and porosity distribution in the matrixes were observed using the JEOL JSM6310 model. All samples were dry before use. Firstly, the sample was frozen in liquid nitrogen for 20-30 seconds and then sectioned using a sharp blade. Then, a specimen plate was coated with a thin layer of gold under 3 mbar pressure for 3-5 minutes with the Edwards Sputter Coater (S150B). The SEM was operated in the range 10-20 kV and micrographs were taken of a number of areas on each sample.

FIGS. 28a-d show SEM micrographs of different samples before any degradation. FIG. 28a shows a Cross section of 85:15 PLGA at 250× magnification. FIG. 28b shows a Cross-section of 75 L: 25 PLGA at 250×magnification. FIG. 28c shows a cross section of a Polymer blend of 65:35 PLA/PCL. FIG. 28d shows a SEM micrograph of 85:15 PLGA at 1000× magnification showing microporous structure.

FIG. 29a-c show SEM micrographs of different samples after 6 weeks of degradation. FIG. 29a shows crystallised 65:35 PLGA, FIG. 29b shows 75 L:25 PLGA and FIG. 29c shows PLGA 85:15.

### Viscosity Test

The viscosity values of the spinning dopes were obtained by using a Bohlin CS 50 Rheometer (Stress Viscometry Model). In order to spin fibres, the viscosity of the polymer solution should be generally between 1-5 Pa·s for non-biodegradable polymers. For biodegradable polymers, the viscosity of the spinning dope might be lower. It is important when applying polymer dope, that the correct amount is used.

### Polymer Degradation Study

0.07 g of each hollow fibre was weighed and immersed in 20 ml of de-ionised water in a screw-topped bottle. These were then placed on an orbital shaker maintained at a temperature of 37° C. The pH of the degradation medium was measured every two days to monitor the rate of generation of acidic byproducts. A Jenway (Model 3051) pH meter was used for this purpose.

### Drug Release Study

In vitro drug release studies were carried out in phosphate buffer saline (PBS) at both room temperature (˜25° C.) and 37° C. PBS solution was applied to keep the degrading system at a constant pH 7.4 value. Every type of polymer fibre was cut into 10 pieces, with 5 cm each. At room temperature, each fibre piece was first sealed at one end with bio-adhesive, and was injected with liquid 5-FU with a syringe pump from the other open end before it was sealed. Every 10 pieces of the same type of fibre were placed into a bottle which was filled with 20 ml of PBS solution. The bottles were placed at room temperature and at a constant temperature water bath, maintained at 37±0.1° C. for 6 months. The amount of drug release was measured every 24 hours within the 3 months. The quantitative measurement of the amount of drug released was measured with a UV Spectrometer. A calibration curve was performed as the concentration versus absorbance for the drug in buffer (wavelength of absorption, □=266 nm (5-FU) and λ=298 nm (cisplatin and carboplatin) or atomic absorption spectroscopy) which was plotted in a logarithmic scale gave a straight line. Samples were drawn from the release medium every 24 hours within the predetermined release period of 3 months to full-fill the cuvette, which would be put into the UV Spectrometer. The absorbance of the drug was noted. The sample in the cuvette was then put back into the release medium.

Hollow fibre membranes of different polymer compositions were investigated to determine their morphology and porosity, response to hydrolytic degradation as well as release rates of the anti-coagulant drug, 5FU and cisplatin and carboplatin.

The following table shows the compositions of the hollow fibres studied and their corresponding spinning conditions.

TABLE 4
Hollow fibres investigated, their compositions and spinning
conditions with range of chemotherapeutics.

| | | | | | Internal |
|---|---|---|---|---|---|
| | | | Polymer/ | Air | Water |
| | | | solvent | Gap | Flowrate |
| No. | Fibre | Composition | wt % | (cm) | (ml/min) |
| 1 | 50:50 PLGA | 50% DL-PLA, 50% PGA | 25 | 0 | 3 |
| 2 | 65:35 PLGA | 65% DL-PLA, 35% PGA | 20 | 0 | 3 |
| 3 | 75:25 PLGA | 75% DL-PLA, 25% PGA | 20 | 0 | 3 |
| 4 | 85:15 PLGA | 85% DL-PLA, 15% PGA | 20 | 0 | 3 |
| 5 | 75L:25 PLGA | 75% L-PLA, 25% PGA | 10 | 0 | 3 |
| 6 | 75:25 PLA/PCL | 75% DL-PLA, 25% PCL | 20 | 0 | 3 |
| 7 | 100 PLA | 100% DL-PLA | 30 | 3 | 4 |
| 8 | 75L:25 PLGA | 75% L-PLA, 25% PGA | 15 | 5 | 4 |
| | | 35% Cisplatin loading | | | |
| 9 | 75L:25 PLGA | 75% L-PLA, 25% PGA | | 5 | 4 |
| | | 30%, 50% and 70% Carboplatin | | | |

### Fluorouracil (5-FU) (see FIG. 30)

Hydrophilic 5-FU accelerates the degradation and drug release rate from polymer hollow fibres as shown in the figure.

Amorphous fibres (PLGA) degrades and releases 5-FU faster than crystalline PLA fibres, 75dL:25PCL has the second slowest degradation and drug release due to its hydrophobicity, but releases twice the 5-FU amount released from PLA fibre.

FIG. 31 shows the percentage of Cisplatin release from hollow fibres of different concentrations where the drug was in liquid form within the lumen of the fibre. FIG. 32 shows the released kinetics of 35% powder carboplatin loaded PLGA 75:25 fibre. FIG. 33 shows the Carboplatin three weeks dose; Percentage mass released (cumulative) of carboplatin against time from carboplatin loaded-fibres fabricated using PLGA (75:25) and 30%, 50% and 70% carboplatin, incubated at 37° C. in PBS (pH 7.4) respectively.

1-66. (canceled)

67. A biocompatible drug delivery device, in which the device is a hollow fibre in which the mean pore size in one or more layers is less than 100 μm, and in which a drug is carried.

68. A hollow fibre as claimed in claim 67, in which the fibre is organic, and in which the fibre comprises a polymer, a binder and one or more drugs.

69. A hollow fibre as claimed in claim 68, in which the polymer is selected from the group consisting of polyethylene, polypropylene, poly(phenylene oxide), polyacrylonitrile, polymethylmethacrylate, poly(vinyl chloride), Poly(vinylidene fluoride), Polyacrylonitrile, Cellulose acetate, Polyamide (aromatic), Polyimide, Poly(ether imide) and poly(vinyl alcohol) co-polymers of Polylactide (PLA) and Polyglycolide (PGA), Polycaprolactone (PCL) and Poly(ethylene terephathalate) (PET), polyhydroxyalkanonate (PHA) class of polymers, poly(imino carbonates) poly(a-hydroxy esters), D-polylactide and L-polylactide, Poly(cyanoacrylates), Biodegradable polyphosphazenes, Pseudo-poly(amino acids), polyethylene glycol containing poly-carbonates, phosphorous containing biodegradable polymers, polyphosphazenes and poly(phosphate esters), natural polymers chitosan and carrageenan

70. A hollow fibre as claimed in claim 68, in which the drug is selected from small molecules, recombinant proteins and anaesthetics, particularly from fluorouracil, cisplatin, oxaliplatin, carboplatin, warfarin and lidocaine.

71. A hollow fibre as claimed in claim 68, in which the fibre has an additional thin outer coating.

72. A hollow fibre as claimed in claim 68, in which the surface area to volume ratio is greater than 1000 m²/m³.

73. A hollow fibre as claimed in claim 68, in which the fibre comprises two or more layers.

74. A hollow fibre as claimed in claim 73, in which the layers are of different compositions or contain different drugs in each layer.

75. A hollow fibre as claimed in claim 74, in which the different compositions have different functionality or affinity for molecules.

76. A method for preparing porous hollow fibres, in which a spinning dope is prepared in a viscous or gel form, sonicated, filtered using a mesh, the dope is degassed in a piston delivery vessel attached to a spinneret, the vessel is pressurized using an inert gas, the dope is extruded through the spinneret to form a fibre precursor, the precursor is washed and dried.

77. A method as claimed in claim 76, in which the spinning dope comprises a polymer or copolymer to increase crosslinking, a solvent and a drug.

78. A method as claimed in claim 77, in which the polymer is selected from the group consisting of polyethylene, polypropylene, poly(phenylene oxide), polyacrylonitrile, polymethylmethacrylate, poly(vinyl chloride), Poly(vinylidene fluoride), Polyacrylonitrile, Cellulose acetate, Polyamide (aromatic), Polyimide, Poly(ether imide) and poly(vinyl alcohol) co-polymers of Polylactide (PLA) and Polyglycolide (PGA), Polycaprolactone (PCL) and Poly(ethylene terephathalate) (PET), polyhydroxyalkanonate (PHA) class of polymers, poly(imino carbonates) poly(a-hydroxy esters), D-polylactide and L-polylactide, Poly(cyanoacrylates), Biodegradable polyphosphazenes, Pseudo-poly(amino acids), polyethylene glycol containing poly-carbonates, phosphorous containing biodegradable polymers, polyphosphazenes and poly(phosphate esters).

79. A method as claimed in claim 77, in which the solvent is selected from the group consisting of ethanol, ethyl acetate or acetone.

80. A method as claimed in claim 77, in which the hollow fibre additionally comprises an affinity agent.

81. Apparatus for the extrusion of a hollow fibre comprising one or more delivery vessels, a spinneret fed by the delivery vessels, a coagulation bath and a washing bath.

82. The use of a hollow fibre as claimed in claim 68 to deliver drugs to treat chronic diseases in mammals.

83. A biocompatible drug delivery device as claimed in claim 67, in which the device is a microsphere in which the mean pore size in one or more layers is less than 100 μm.

84. A microsphere as claimed in claim 83 in which the fibre has a mean pore size in the one or more layers of less than 50 μm, less than 1 μm, less than 10 nm, or less than 10 nm.

85. A microsphere as claimed in claim 84, in which the polymer is selected from the group consisting of polyethylene, polypropylene, poly(phenylene oxide), polyacrylonitrile, polymethylmethacrylate, poly(vinyl chloride), Poly(vinylidene fluoride), Polyacrylonitrile, Cellulose acetate, Polyamide (aromatic), Polyimide, Poly(ether imide) and poly(vinyl alcohol) co-polymers of Polylactide (PLA) and Polyglycolide (PGA), Polycaprolactone (PCL) and Poly(ethylene terephathalate) (PET), polyhydroxyalkanonate (PHA) class of polymers, poly(imino carbonates) poly(a-hydroxy esters), D-polylactide and L-polylactide, Poly(cyanoacrylates), Biodegradable polyphosphazenes, Pseudo-poly(amino acids), polyethylene glycol containing poly-carbonates, phosphorous containing biodegradable polymers, polyphosphazenes and poly(phosphate esters)

86. A microsphere as claimed in claim 84 in which the drug is selected from small molecules, recombinant proteins and anaesthetics, in particular from fluorouracil, cisplatin, oxaliplatin, carboplatin, warfarin and lidocaine.

87. A microsphere as claimed in claim 84, in which there is additionally an agent to create an emulsion.

88. A microsphere as claimed in claim 83, in which the microspheres have a high loading of drugs.

89. A microsphere as claimed in claim 88, in which the drugs are present in an amount of at least 10% by weight, or of at least 20%, 30%, 40%, 50%, 60% or 70% by weight.

90. A method for preparing microspheres, in which a polymer is dissolved in a suitable solvent; a drug is dissolved in or dispersed in an organic solvent containing the polymer; and the mixture is fed to:
(i) a high pressure airbrush or spray device with a small nozzle; and the mixture is sprayed under water and microspheres are formed by solvent de-mixing; or
(ii) a ceramic hollow fibre bundle with 2-20 nm pores to create microdroplets under water, and microspheres are formed by solvent de-mixing; or
(iii) a high pressure airbrush with a small nozzle; and the mixture is sprayed into an antistatic chamber with water saturated air and the solvent is extracted from the produced droplets to form microspheres.
